(19) United States
(12) Patent Application Publication
(10) Pub. No.: US 2005/0090518 A1
(43) Pub. Date: Apr. 28, 2005

(54) Method for treating parkinson's disease using apomorphine and apomorphine prodrugs

(75) Inventor: Steven C. Quay, Edmonds, WA (US)

Correspondence Address: Nastech Pharmaceutical Company Inc., 3450 Monte Villa Parkway, Bothell, WA, 98021-8906, US

(73) Assignee: Nastech Pharmaceutical Company Inc.

(21) Appl. No.: 10/964,327

(22) Filed: Oct. 12, 2004

Related U.S. Application Data
(60) Provisional application No. 60/514,382, filed on Oct. 24, 2003.

(51) Int. Cl. A61K 31/4745; A61K 31/7052

(52) U.S. Cl. 514/289; 514/23

(57) ABSTRACT

A formulation and method for treating dopamine deficiency such as occurs in Parkinson's disease in a mammal wherein apomorphine is administered in conjunction with an apomorphine prodrug. Preferably the apomorphine prodrug is a diester of apomorphine and the mammal is a human.

This claims the benefit under 35 U.S.C. §119 (e) of U.S. Provisional Patent Application No. 60/514,382 filed on Oct. 24, 2003 the entire contents of which are incorporated herein by reference.

### BACKGROUND OF THE INVENTION

The teachings of all of the references cited herein are incorporated in their entirety by reference.

Parkinsonism or Parkinson's disease is a neurodegenerative disorder consisting of a variable combination of tremor, muscular rigidity, bradykinesia (slowness and poverty of movement), and an impairment of postural balance leading to disturbances of gait and falling. Parkinson's disease is a chronic, progressive disorder in which idiopathic parkinsonism occurs without evidence of more widespread neurological involvement.

Parkinson's disease generally commences in middle or late life and leads to progressive disability with time. The disease occurs in all ethnic groups, has an equal sex distribution, and is common, with a prevalence of 1 to 2 per 1000 of the general population and 2 per 100 among people over 65 years. Signs of parkinsonism are extremely common in the elderly. It is estimated that 15% of individuals between 65 and 74 years of age, and more than half of all individuals after age 85, have abnormalities on examination consistent with the presence of an extrapyramidal disorder.

Symptoms of Parkinson's disease are caused by loss of nerve cells in the pigmented substantia nigra pars compacta and the locus coeruleus in the midbrain. Cell loss also occurs in the globus pallidus and putamen. Eosinophilic intraneural inclusion granules (Lewy bodies) are present in the basal ganglia, brainstem, spinal cord, and sympathetic ganglia.

Pars compacta neurons of the substantial nigra provide dopaminergic input to the striatum, which is part of the basal ganglia. In Parkinson's disease, loss of pars compacta neurons leads to striatal dopamine depletion and ultimately to reduced thalamic excitation of the motor cortex. Other neurotransmitters, such as norepinephrine, are also depleted, with clinical consequences that are uncertain but perhaps contribute to depression, dysautonomia, and “freezing” episodes of marked akinesia.

The cause of Parkinson's disease is unknown. One suggested cause is exposure to an unrecognized environmental toxin, perhaps structurally similar to 1-methyl-4-phenyl-1,2,3,6-tetrahydropyridine (MPTP). Alternatively or additionally, endogenous toxins may be responsible. In particular, the normal neurotransmitter dopamine readily oxidizes to produce free radicals, which can cause cell death.

Oxidative stress is likely when dopamine turnover is increased, glutathione is reduced, (leaving neurons more vulnerably to oxidant stress), and reactive iron is increased (promoting the generation of potentially toxic hydroxyl radicals). A mitochondrial complex 1 defect occurs in Parkinson's disease and may contribute to neuronal vulnerability and loss through free radical generation.

Current therapies include the administration of anticholinergic drugs in the early stages of treatment and to patients under 60 years of age when no functional impairment is present. Commonly used anticholinergics include benztropine 0.5 to 2 mg orally 3 times a day and trihexyphenidyl 2 to 5 mg orally 3 times a day. Antihistamines with anticholinergic action such as diphenhydramine, 25 to 200 mg/day orally and orphenadrine 50 to 200 mg/day orally are useful for treating tremor. Anticholinergic tricyclic antidepressants such as amitriptyline 10 to 150 mg orally at bedtime often are useful in treating depression associated with Parkinson's disease.

If functional impairment is present or the patient is 60 years of age or older, then amantadine is administered, 100 to 300 mg/day orally alone or in conjunction with the anticholinergics or levodopa.

As the disease progresses, the drug of choice is levodopa (L-DOPA, LARODOPA, DOPAR, L-3,4-dihydroxyphenylalanine), the metabolic precursor to dopamine.

Dopamine replacement therapy is used in Parkinson's disease to restore the brain's natural dopamine which is lost as the neurons in the substantia nigra degenerate. Pharmacologic therapy for Parkinson's disease includes use of levodopa, a dopamine precursor, to combat symptoms of akinesia and rigidity. Levodopa readily crosses the blood brain barrier where it is converted by dopamine carboxylase into dopamine. Coadministration of the peripheral decarboxylase inhibitor carbidopa or benserazide inhibits peripheral decarboxylase and lowers dosage requirements by preventing catabolism and also reduces nausea produced when dopamine is released into the circulation by peripheral conversion of levodopa to dopamine. In most individuals, a daily dose of 75 mg of carbidopa is sufficient to prevent the development of nausea. A commonly prescribed form of carbidopa/levodopa is the form containing 25 mg of carbidopa and 100 mg of levodopa (SINEMET, ATAMET). Other formulations contain the following ratios of carbidopa and levodopa respectively 10 mg /100 mg, 25 mg/250 mg and in a controlled-release tablet, 50 mg/200 mg.

The combination of levodopa/carbidopa also allows a lower total dose of levodopa to be administered, which results in a lower rate of side effects, particularly nausea and vomiting. These side effects are believed to be caused by peripheral conversion of levodopa to dopamine. Another useful adjunct to levodopa therapy is the use of catechol O-methyltransferase inhibitors such as tolcapone and entacapone, which inhibit the breakdown of dopamine.

Because the half-life of levodopa/carbidopa combination is only about 1.5 hours, the combination is typically given three to four times daily. A sustained release preparation is available which is typically given two or three times daily. However, with this formulation, the onset to action is slower, which may be a problem particularly with the first dose in the morning. Another group of therapeutic agents that are used as alternative to levodopa are the dopamine-receptor agonists including bromocriptine, pergolide, ropinirole and pramipexole. Since enzymatic conversion of these drugs is not required for activity, they do not depend on the functional capacities of the nigrostriatal neurons and thus might be more effective than levodopa in late Parkinson's disease. In addition, dopamine-receptor agonists potentially are more selective in their actions. Levodopa activates all dopamine receptor types throughout the brain, agonists may exhibit relative selectivity for different subtypes of dopamine receptors. Bromocriptine and pergolide are both ergot derivatives and share a similar spectrum of therapeutic actions and adverse effects. Bromocriptine is strong agonist of the D₂ class of dopamine receptors and a partial antagonist of the D1 receptors, while pergolide is an agonist of both classes. Ropinirole and pramipexole have selective activity at D₂ class sites (specifically, at the D₂ and D₃ receptor proteins) and little or no activity at D₁ class sites.

Unfortunately, over time, dopamine/carbidopa therapy loses its beneficial effects. With time, fluctuations or variations in the effect of dopamine/carbidopa therapy result in “on-off” response, where movement may suddenly be inhibited and the patient will “freeze.” It has been hypothesized that these “on-off” responses may be a result of an overall decreased efficacy or of variability of gastrointestinal absorption of the dopamine/carbidopa combination. Ultimately, new movement abnormalities such as akinesia (immobility) and dyskinesias or an alternation between involuntary movements and immobility often ensue. With time, other side effects also may occur, including hallucinations. The treatment for such neuropsychological side effects is interruption of the dopamine/carbidopa therapy, making movement disorders more severe.

In summary, the current pharmacologic therapy of Parkinson's disease has several limitations, including:
The effectiveness of levodopa/carbidopa decreases over time
New movement abnormalities such as the “on-off” response and immobility occur with therapy
Variations in gastrointestinal absorption may play a role in the variable response

Thus, there is a need to provide for a new class of therapeutics that can act as agonist to the D2 class of dopamine receptors, and give immediate and long-lasting alleviation of the symptoms of Parkinson's disease.

### DESCRIPTION OF THE OF INVENTION

The present invention fills this need by providing for a formulation comprised of apomorphine and a prodrug homolog of apomorphine capable of providing for a sustained release of apomorphine. A prodrug of apomorphine is a derivative or homolog of apomorphine that can be metabolized in the body to produce apomorphine. Examples of apomorphine prodrugs that can be used according to the present inventions, are apomorphine diesters or diacylapomorphines as disclosed in U.S. Pat. No. 4,080,456.

The present invention is further directed to a method of treating a dopamine deficiency, such as Parkinson's disease, in a mammal comprised of administering a prodrug of apomorphine in conjunction with apomorphine to said mammal. Preferably the mammal is human, but other mammals such as dogs, cats, other primates, horses etc. can also be treated according to the method of the present invention.

Apomorphine, as a dopamine agonist, has been proposed as a therapy for Parkinson's disease since at least 1970 and as a therapy for “on-off” fluctuations since at least 1987.
Apomorphine

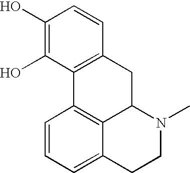

Routes of administration have included subcutaneous, sublingual, intranasal, and continuous infusion intravenous. Subcutaneous and continuous infusion require injection, which risks local infection, patient discomfort, needle sticks to the caregiver, as well as injection site reactions. The bioavailability of the subcutaneous formulation is limited to approximately 4%. Therefore, intranasal administration is a promising alternative. Recent studies describe good efficacy (95% abolition of “off state” events) and fast onset of action (10 minutes) following intranasal administration. However, the duration of efficacy is limited to less than one hour.

Apomorphine or its variants can be administered orally, sublingually, subcutaneously or intranasally. A sublingual or oral dose of an apomorphine should be about 2-10 mg. However, if the apomorphine is administered intranasally the dose should range from about 2 mg 4mg (See U.S. Pat. No. 6,436,950). Other dopamine receptor agonists can be administered at the following doses: bromocriptine mesylate (Geneva Pharmaceuticals, Broomfield, Colo.) 2.5-10 mg, ropinirole (REQUIP®,GlaxoSmithKline, Research Triangle Park, N.C.) 0.25-5 mg, cabergoline (DOSTINEX®, Pharmacia & Upjohn, Peapack, N.J.) 0.5-7 mg, and pramipexole dihydrochloride (MIRAPEX®, Pharmacia & Upjohn) 0.125-1.5 mg.

A preferred apomorphine hydrochloride hemihydrate nasal formulation is comprised of the following as percent of total weight (% w/w):

| | | |
|---|---|---|
| Apomorphine Solution | Apomorphine HCl, USP | 1 |
| | Citric Acid Anhydrous, USP | 0.69 |
| | Sodium Citrate Dihydrate, USP | 0.42 |
| | Propylene Glycol, USP | 7 |
| | Glycerin, USP | 4.98 |
| | Ascorbic Acid, USP | 0.012 |
| | Sodium Metabisulfite, NF | 0.088 |
| | Edetate Disodium, USP | 0.02 |
| | Benzalkonium Chloride, USP | 0.04 |
| | Sodium Hydroxide, NF (1 N) | TAP |
| | Hydrochloric Acid, NF (1 N) | TAP |
| | Purified water q.s. | 100 |

To counter the short term efficacy of apomorphine in Parkinson's disease, investigators as early as 1976 began the synthesis of diesters of apomorphine. The goal was to design a prodrug which will be slowly converted to apomorphine, thus allowing a single dose to retain its effects for a longer period. Examples of such apomorphine diesters are disclosed in U.S. Pat. No. 4,080,456, such as dipropionylapomorphine, di-n-butrylapomorphine, diisobutyrylapomorphine and divaleroylapomorphine. Other prodrugs of apomorphine that can be used according to the process of the present invention are the glycoside and orthoester glycoside derivatives of apomorphine as disclosed in International Patent Publication No. WO 03/080074 A1. According to the present invention the apomorphine prodrug such as the diesters are administered in conjunction with apomorphine. Due to their central nervous system activity, the apomorphine prodrugs and apomorphine are useful as tremor-reducing agents in human and veterinary medicine. The apomorphine prodrugs and apomorphine together are symptomatically effective in the treatment of motor disorders of the type associated with dopamine deficiency by the administration of a catalepsy-abolishing effective amount of an apomorphine prodrug in conjunction with apomorphine. The apomorphine will give immediate relief, while the apomorphine prodrug will give a sustained released amelioration of the symptoms associated with dopamine deficiency of the extrapyramidal system of the CNS, as occurs in Parkinson's disease.

The apomorphine prodrugs can be administered in a mixture with conventional excipients. The apomorphine prodrug can be administered in a mixture containing apomorphine, or the prodrug can be administered separately. The apomorphine prodrugs are generally administered to animals, including but not limited to humans. A tremor-reducing effective daily dosage of the active apomorphine prodrug as administered orally to humans generally comprises 0.1 to 100, preferably 1 to 10 mg/kg of body weight. Preferably the amount ratio of the apomorphine prodrug to apomorphine present in the formulation is about 2 to 1. For example, 2 mg of apomorphine is administered with 4 mg of the apomorphine prodrug.

The apomorphine prodrugs are preferentially administered intranasally in a formulation that also contains apomorphine. Such intranasal formulations can be made according to the procedures described by Illum et al. in U.S. Pat. No. 6,342,251, by Merkus, F. in U.S. Pat. No. 5,756,483, and by Achari et al. in U.S. Pat. No. 6,436,950.

Examples of other apomorphine diesters are:

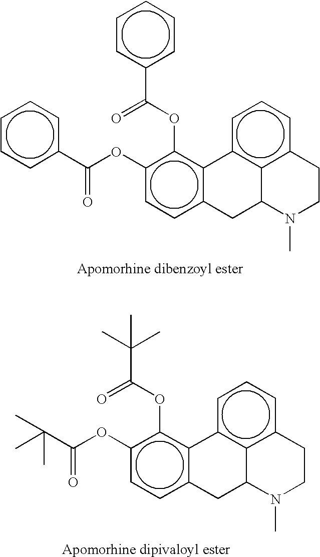

| | % w/w |
|---|---|
| Dibenzoyl apomorphine | 4.00 |
| Lactic Acid (0.2 M) | 50.0 |
| Propylene Glycol, USP | 40.0 |
| Sodium Metabisulfite, NF | 0.10 |
| Benzalkonium Chloride (50% soln), | 0.04 |
| NF | |
| Edetate disodium, USP | 0.02 |
| Purified Water, USP, qs | 100.0 |

Sample 2

| | % w/w |
|---|---|
| Dibenzoyl apomorphine | 3.5 |
| Lactic Acid (0.2 M) | 63.0 |
| Propylene Glycol, USP | 30.0 |
| Sodium Metabisulfite, NF | 0.10 |
| Benzalkonium Chloride (50% soln), | 0.04 |
| NF | |
| Edetate disodium, USP | 0.02 |
| Purified Water, USP, qs | 100.0 |

Sample 3 Dibenzoyl apomorphine in Labrafac® CC (40mg drug in 1 g) Note: Labrafac® CC is a medium chain triglyceride, immiscible with water.
Sample 4 Dibenzoyl apomorphine in Labrasol® and 0.2M Lactic Acid combination Note: Labrasol® is composed of a mixture of mono-, di- and triglycerides and mono- and di- fatty acid esters of polyethylene glycol.
Apomorphine disuccinic d-glucosamine

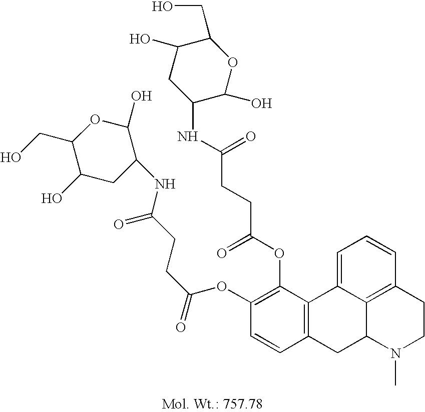

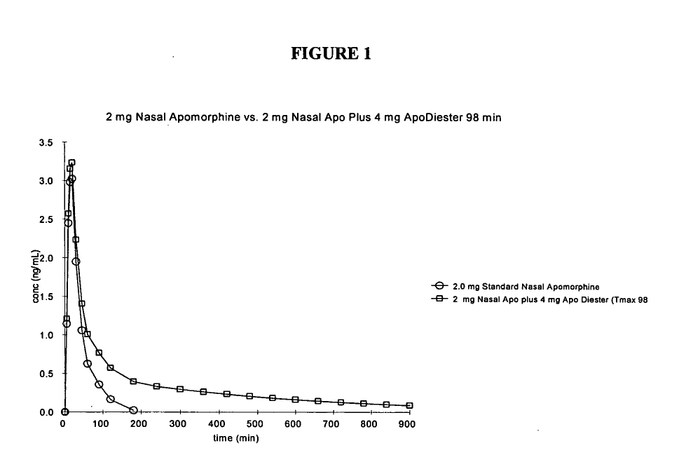

The combination of immediate release apomorphine with a sustained release diester prodrug of apomorphine can be modeled as in FIG. 1. The graph shows that to sustain a plasma apomorphine level of 0.5 ng/mL, immediate release apomorphine lasts approximately 50 minutes, whereas the combination of immediate release and sustained release lasts approximately 100 minutes. To sustain a plasma apomorphine level of 0.25 ng/mL, immediate release apomorphine lasts approximately 100 minutes, whereas the combination of immediate release and sustained release lasts approximately 400 minutes. To prevent “on-off” fluctuations, it is believed that the apomorphine plasma levels should be in the 0.25 -0.5 ng/mL range. By selection of the hydrolysis rate for the apomorphine diester, and by adjusting the relative concentrations of apomorphine vs apomorphine diester in the combination product, it is proposed that a formulation be developed which maintains plasma apomorphine concentrations above the “clinical threshold” for 6-8 hours. This will result in a once per day dosage form.

1. A formulation for treating a dopamine deficiency in the central nervous system in a mammal comprised of apomorphine and an apomorphine prodrug.

2. The formulation of claim 1 wherein the apomorphine prodrug is selected from the group consisting of apomorphine diester, diacylapomorphines, glycoside derivatives of apomorphine and orthoester glycoside derivatives of apomorphine.

3. The formulation of claim 2 wherein the apomorphine prodrug is selected from the group consisting of dipropionylapomorphine, di-n-butyrylapomorphine, diisobutyrylapomorphine and divaleroylapomorphine.

4. The formulation of claim 1 wherein the formulation is a formulation for treating Parkinson's disease.

5. The formulation of claim 1 wherein the formulation is suitable for intranasal administration.

6. A method for treating a mammal having a dopamine deficiency in the central nervous system comprised of administering to said mammal therapeutically effective amounts of apomorphine in conjunction with an apomorphine prodrug.

7. The method of claim 6 wherein the apomorphine prodrug is selected from the group consisting of apomorphine diester, diacylapomorphines, glycoside derivatives of apomorphine and orthoester glycoside derivatives of apomorphine.

8. The formulation of claim 7 wherein the apomorphine prodrug is selected from the group consisting of dipropionylapomorphine, di-n-butyrylapomorphine, diisobutyrylapomorphine and divaleroylapomorphine.

9. The method of claim 6 wherein the mammal has Parkinson's disease.

10. The method of claim 9 wherein the mammal is human.

11. A method for alleviating an “on-off’ episode in an individual afflicted with Parkinson's disease comprising administering apomorphine in conjunction with an apomorphine prodrug.

12. The method of claim 11 wherein the apomorphine prodrug is selected from the group consisting of apomorphine diester, diacylapomorphines, glycoside derivatives of apomorphine and orthoester glycoside derivatives of apomorphine.

13. The method of claim 12 wherein the apomorphine prodrug is selected from the group consisting of dipropionylapomorphine, di-n-butyrylapomorphine, diisobutyrylapomorphine and divaleroylapomorphine.

14. The method of claim 11 wherein the individual is human.
